SEQUENCE LISTING (15 sequences)

SEQ ID NO: 1 - DNA, 500 bp, synthetic construct
  source 1..500: mol_type = other DNA
aggtacacaccaaggccaaccacatcaacttgtttatcacttcaatcactcttctctcggccttggtctcccctctaaccaccgtgatttgtactaacacaacagccaatgacgtgaacaatgcaattgcattcaatatgaagaatatcttaaacgacgtggtgttcaccatcaccgccattccagagtcgtcgtcgcctctagggacggtgaagatagcgtcaaaagccaccgttgcaaagaggaaaaccacaactgtgactgagtggcgttgttgattcctgccctgtgcagtttgcgaagctctttggcgcgtttttgttggtttttcgggtttgttctagctggatgtgtacgtcctttttgatctgtgtcactgtttttcttagctcgtctcgcggttggttgagctatttggcttttacgccgccatatcgagctagagtttcctttattagtgatgtttcttcgcatagtgtaaggccttcggctatgtcgaa

SEQ ID NO: 2 - DNA, 500 bp, synthetic construct
  source 1..500: mol_type = other DNA
tcggatctgttagacaaccttaacttggagcattagctaccttcattttcctcagaggtttgttggcagtgttcgtgttcttacgtgtagtagttggagtggtctttcttggagtatctcctgtgaaaccttttcatttgtccctattgctattattatcataaggaatagatttatcacgaatttcttcatcttgagccatattctctagcagaagtctagcctccagttggctaagcttgcatcctgcgagaaaattcttcttgtgctgccaattttagcttgattttggcgagttctttggccatatcaacgactgtaggatttggcttgcaataataatcatctttttctttttctttcccaccatcttcagggtcttcgtcttcggaacattcctctcgaccctcattgatgggcttttcatcaacataattttcattacgttgagtctttttggcaggatgaatagctgcttcagttggatctctgttgactct

SEQ ID NO: 3 - DNA, 500 bp, synthetic construct
  source 1..500: mol_type = other DNA
tttgatgaagcacaaatggcaaacctctttgctagtaattgagttaacccaactacaaagttagtgacaatctcggaggtgaatggagccaagacagacccaacgtagttagagaaccagtcgttcatggggtccaaatttaagttcaaatttccttgacatactcctccttgcgagctacatcctgatgctcattaatccaacgcttcaggacatcagattccgcagaccccttagtctgggcgtccatagaaaaattgtgtcgggccaaacagagtgcaatatgtttagggtatgagtgaacatgagtcgaaggaattgagggaacaggttggaattgctcagctttagaaggaatgctggaattgcgggtgccctgagagggagccttggtggatttagccttcttcaaagaagactctacaggagccctctccttcctttttcctttcctatagactaggggttacatcccgctccttgagcacaaccacctccaa

SEQ ID NO: 4 - DNA, 12 bp, synthetic construct
  source 1..12: mol_type = other DNA
ccacaactgtga

SEQ ID NO: 5 - skipped

SEQ ID NO: 6 - DNA, 24 bp, synthetic construct
  source 1..24: mol_type = other DNA
tagaaaaattgtgtcgggccaaac

SEQ ID NO: 7 - DNA, 22 bp, synthetic construct
  source 1..22: mol_type = other DNA
aagaggaaaaccacaactgtga

SEQ ID NO: 8 - DNA, 21 bp, synthetic construct
  source 1..21: mol_type = other DNA
ccaccgttgcaaagaggaaaa

SEQ ID NO: 9 - DNA, 20 bp, synthetic construct
  source 1..20: mol_type = other DNA
actctagctcgatatggcgg

SEQ ID NO: 10 - DNA, 21 bp, synthetic construct
  source 1..21: mol_type = other DNA
ctaagcttgcatcctgcgaga

SEQ ID NO: 11 - DNA, 22 bp, synthetic construct
  source 1..22: mol_type = other DNA
agttggctaagcttgcatcctg

SEQ ID NO: 12 - DNA, 21 bp, synthetic construct
  source 1..21: mol_type = other DNA
tgagggtcgagaggaatgttc

SEQ ID NO: 13 - DNA, 20 bp, synthetic construct
  source 1..20: mol_type = other DNA
ccaacgcttcaggacatcag

SEQ ID NO: 14 - DNA, 20 bp, synthetic construct
  source 1..20: mol_type = other DNA
aattgtgtcgggccaaacag

SEQ ID NO: 15 - DNA, 20 bp, synthetic construct
  source 1..20: mol_type = other DNA
ccgcaattccagcattcctt

(19) United States
(12) Patent Application Publication
(10) Pub. No.: US 2026/0117323 A1
(43) Pub. Date: Apr. 30, 2026

(54) METHOD TO DEVELOP DIAGNOSTIC MOLECULAR MARKERS TO IDENTIFY HAPLOID PLANTS IN ONE-SHOT AMPLIFICATION

(71) Applicant: Haplotype Holdings Inc., Las Vegas, NV (US)

(72) Inventor: Andrew Phengrasmy, Las Vegas, NV (US)

(21) Appl. No.: 19/371,145

(22) Filed: Oct. 28, 2025

Related U.S. Application Data
(60) Provisional application No. 63/712,881, filed on Oct. 28, 2024.

(51) Int. Cl. C12Q 1/6895; C12Q 1/6806; C12Q 1/686; C12Q 1/6869

(52) U.S. Cl. CPC C12Q 1/6895; C12Q 1/6806; C12Q 1/686; C12Q 1/6869; C12Q 2600/13; C12Q 2600/156; C12Q 2600/16

(57) ABSTRACT

The present invention relates to a rapid and accurate method for identifying haploid plants derived from crosses between Cannabis donor lines and natural inducer lines. The method comprises: sequencing maternal and paternal lines using whole genome sequencing (WGS); identifying contrasting molecular markers between maternal and paternal lines; developing diagnostic primers targeting the molecular marker; and screening F1 progeny with one-shot PCR to detect genotype indicative of haploidy.

### CROSS-REFERENCE TO RELATED APPLICATIONS

This application claims the benefit of U.S. Provisional Patent Application No. 63/712,881 filed Oct. 28, 2024 which is incorporated by reference herein in its entirety.

### REFERENCE TO SEQUENCE LISTING SUBMITTED ELECTRONICALLY

This application contains a sequence listing, which is submitted electronically. The information contained in the electronic sequence listing (070500-2US2 Sequence Listing.xml; size: 15,103 bytes; and date of creation: Oct. 27, 2025) is incorporated herein by reference in its entirety.

### BACKGROUND

The present invention relates to the field of plant breeding and, more specifically, to the identification of haploid plants obtained through in vivo haploid induction. In doubled haploid (DH) technology, the rapid and accurate discrimination between haploids and diploids is a crucial step for the efficient production of homozygous lines. Traditional identification methods based on visual markers, morphological traits, or cytological analyses present significant limitations, including genotype dependency, environmental influence, labor-intensive procedures, and limited scalability. These constraints are particularly relevant in Cannabis sativa and related species, where natural pigmentation patterns and regulatory restrictions hinder the adoption of conventional markers. There is therefore a need for a fast, reliable, and genotype-dependent molecular approach that can identify haploid plants at early developmental stages, reduce false positives, and integrate seamlessly into high-throughput breeding pipelines.

Plants naturally produce haploids at a low frequency, and the identification of haploid individuals is a critical step in doubled haploid (DH) technology, enabling the rapid development of completely homozygous lines for plant breeding (Deng et al., Critical Reviews in Plant Sciences. 2024). Haploid development through androgenesis occurs in cannabis (Galán-Ávila et al., Front. Plant Sci. 2021), however, these haploid plants are not stable (Ahsan et al., Biology 2025; Tonolo, bioRxiv. 2024). In maize (Zea mays L.). Numerous tools have been developed to discriminate haploids from diploids, ranging from manual selection based on morphological traits to cytological and molecular diagnostics, with varying degrees of accuracy, scalability, and cost-effectiveness (Chaikam et al., Theor. Appl. Genet. 2015; Melchinger et al., Crop Sci. 2016. Haploid can be identified through visual selection using phenotypic biomarkers integrated into haploid inducer lines. The most widely used biomarker is the R1-navajo (R1-nj) anthocyanin trait, which produces purple pigmentation in the aleurone and scutellum, allowing discrimination at the seed stage (Nanda and Chase, Crop Sci. 1966; Chaikam et al., Theor. Appl. Genet. 2015). Similarly, the Pl-1 red root gene can generate anthocyanin pigmentation in seedling roots (Rotarenco et al., Maize Genet. Coop. News Lett. 2010), whereas the high kernel oil content (KOC) marker exploits the xenia effect of high-oil pollen to distinguish diploid and haploid kernels by using near-infrared spectroscopy (Melchinger et al., Sci. Rep. 2013). Fluorescent markers such as GFP or DsRED, alone or in dual systems, have also been engineered into inducer genotypes, enabling visual screening of seeds or seedlings under specific light conditions (Yu and Birchler, Mol. Breed. 2016; Dong et al., Mol. Plant. 2018). These biomarker-based systems are practical and non-destructive but can suffer from reduced accuracy due to genetic background effects, marker suppression by inhibitor alleles, or natural pigmentation in the donor germplasm (Prigge et al., Crop Sci. 2011; Gain et al., Mol. Biol. Rep. 2023).

Beyond pigmentation, other morphological and physiological differences between haploids and diploids can be exploited at various growth stages. Haploids generally display reduced vigor, shorter plant stature, narrower leaves, smaller internode diameters, and earlier flowering compared to diploids (Chase, Am. J. Bot. 1964; Chalyk, Euphytica. 1994). Such differences can also be detected in seedlings within days after germination, with haploids exhibiting shorter radicle and coleoptile lengths, fewer seminal roots, and reduced growth rates (Chaikam et al., 2015; Vanous et al., Plant Phenome J. 2019). While these traits can be useful for early screening, they are strongly influenced by genotype and environment and often require population-specific threshold calibration (Baleroni et al., Crop Breed. Appl. Biotechnol. 2021).

Cytological methods provide gold-standard verification of ploidy levels. Chromosome counting in root tip cells or meiocytes offers direct determination of chromosome number (Kiesselbach and Petersen, Genetics. 1925; Doležel et al., Nat. Protoc. 2007), but is laborious, time-consuming, and requires specialized cytogenetic expertise. Flow cytometry, by contrast, allows rapid and accurate estimation of nuclear DNA content in large sample sets, distinguishing haploids from diploids within minutes (De Laat et al., Planta. 1987; Cousin et al., Cytom.A: J. Int. Soc Anal. Cytol. 2009; Molenaar et al., Plant Breed. 2019). This method has become the primary standard for confirming putative haploids selected via preliminary screening.

Molecular marker-based genotyping instead offers high accuracy and flexibility across genetic backgrounds. Codominant markers such as simple sequence repeats (SSR) and single nucleotide polymorphisms (SNP) can be used to distinguish haploids, which carry only maternal alleles, from diploids, which are heterozygous for both maternal and paternal alleles (Xu et al., J. Exp. Bot. 2013; Semagn et al., Mol. Breed. 2014). SSRs have been widely applied but require prior polymorphism screening between parents and are less amenable to high-throughput workflows. SNP-based assays, particularly when integrated into low-cost platforms such as Kompetitive Allele-Specific PCR (KASP) or TaqMan, can deliver rapid, scalable, and cost-efficient haploid identification, sometimes using only a single polymorphic locus (Kelliher et al., Nature. 2017; Khammona et al., Front. Plant Sci. 2024). In practice, many breeding programs adopt multi-tiered strategies, combining a rapid preliminary selection (e.g., R1-nj or KOC) with a more accurate secondary confirmation (e.g., flow cytometry or molecular markers) to balance throughput, cost, and reliability. This stratified approach increases overall efficiency, reduces false positives, and ensures high-quality DH production pipelines (Chaikam et al., Theor. Appl. Genet. 2015; Melchinger et al., Crop Sci. 2016).

### SUMMARY OF THE INVENTION

The present invention relates to a rapid and accurate method method for screening a Cannabis plant obtained by crossing an inducer genotype with a commercial variety for haploidy, wherein the method comprises:
a) sequencing maternal and paternal lines using whole genome sequencing (WGS);
b) identifying contrasting molecular markers between maternal and paternal lines;
c) developing diagnostic primers targeting the molecular marker;
d) screening F1 progeny with one-shot PCR to detect genotype indicative of haploidy.

In some embodiments, the plant is Cannabis accessions (Sativa, Indica, and Ruderalis).

In some embodiments, the molecular marker is a Single Nucleotide Polymorphism (SNP) marker.

In some embodiments, the molecular marker is an Insertion/Deletion (InDel) marker.

In some embodiments, the molecular marker is a SNP marker and an InDel marker.

In some embodiments, the InDel is at least 6 nucleotides in length.

In some embodiments, the molecular markers are distributed across different chromosomes.

In some embodiments, no additional SNPs or InDels are identified at least 50 nucleotides upstream and downstream of the molecular marker.

In some embodiments, the molecular marker specific to the maternal line is located on chromosome 10, and it is represented by Marker ID 1 (SEQ ID NO: 1).

In some embodiments, the molecular marker specific to the maternal line is located on chromosome 6, and it is represented by Marker ID 2 (SEQ ID NO: 2).

In some embodiments, the molecular marker specific to the paternal line is located on chromosome 3, and it is represented by Marker ID 3 (SEQ ID NO: 3).

In some embodiments, the diagnostic primers are selected from the primer pairs as defined in Table 1.

In some embodiments, the method further comprises confirming the putative haploid Cannabis plant utilizing DNA sequencing or flow cytometry.

Further aspects, features and advantages of the present invention will be better appreciated upon a reading of the following detailed description of the invention and claims.

### BRIEF DESCRIPTION OF THE DRAWINGS

The foregoing and other objects, aspects, features, and advantages of exemplary embodiments will become more apparent and may be better understood by referring to the following description taken in conjunction with the accompanying drawings.

FIGS. 1A-1C shows the identification of contrasting InDels between the haploid inducer line and the commercial Cannabis variety which serve as the female line by whole genome sequencing. Marker ID 1 identifies a deletion present in the paternal haploid inducer line KA-5 (annotated as S14) and absent in the maternal commercial variety S33 (annotated as S15), located on chromosome 10 at positions 58,833,639-58,833,678 bp (FIG. 1A). Marker ID 2 identifies a deletion present in the paternal line and absent in the maternal line, located on chromosome 6 at positions 85,669,285-85,669,324 bp (FIG. 1B). Marker ID 3 identifies a deletion present in the maternal commercial variety and absent in the male haploid inducer line, located on chromosome 3 at positions 22,447,069-22,447,108 bp (FIG. 1C). Grey bars represent aligned sequencing reads; black gaps indicate deletions; letters represent SNPs in the alignment. The contrasting alleles at these loci enable the design of diagnostic InDel-based PCR markers for discrimination between maternal and paternal genotypes.

FIGS. 2A-2C shows PCR amplification of diagnostic InDel markers in the male line (KA-5), commercial Cannabis variety (S33), and negative control (−). Amplification of Marker ID 1 is observed only in the commercial Cannabis variety (S33), consistent with the presence of the deletion in the male line (KA-5) (FIG. 2A). Amplification pattern of Marker ID 2 confirms the presence of the deletion in KA-5 and absence in the commercial Cannabis S33 (FIG. 2B). Amplification of Marker ID 3 is observed only in KA-5, consistent with the deletion present in the maternal variety S33 (FIG. 2C). Lane M: molecular size marker 1 kb.

FIG. 3 shows PCR-based screening of F1 progeny using the combination of InDel diagnostic markers. Each lane corresponds to a different F1 individual obtained from the cross between the male haploid parent KA-5 and the female commercial Cannabis variety S33. Diploid individuals amplify both maternal and paternal markers, indicating the presence of genetic material from both parents. Putative haploids amplify only maternal-specific markers and show no amplification for paternal-specific markers, consistent with the absence of paternal alleles. Lane M: molecular size marker. Lane H: haploid line with maternal-specific amplicon.

FIG. 4 shows flow cytometry analysis confirming ploidy level in Cannabis F1 progeny. (Left) Representative histogram of a diploid control showing the main G1 peak corresponding to 2C DNA content. (Center) Representative histogram of a haploid individual showing a main G1 peak corresponding to 1C DNA content. (Right) Overlay of flow cytometry histograms from multiple diploid and haploid individuals, illustrating the clear shift in DNA content between the two ploidy classes. Peaks correspond to nuclei populations at different cell cycle stages (C1, C2, C4).

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Although any methods and materials similar or equivalent to those described herein can be used in the practice for testing of the present application, exemplary materials and methods are described herein.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications, and publications cited herein are incorporated by reference as if set forth fully herein. It must be noted that as used herein and in the appended claims, the singular forms “a,” “an,” and “the” include plural reference unless the context clearly dictates otherwise.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Unless otherwise stated, any numerical value, such as a concentration or a concentration range described herein, are to be understood as being modified in all instances by the term “about.” Thus, a numerical value typically includes ±10% of the recited value. For example, a dosage of 10 mg includes 9 mg to 11 mg. As used herein, the use of a numerical range expressly includes all possible subranges, all individual numerical values within that range, including integers within such ranges and fractions of the values unless the context clearly indicates otherwise.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Throughout this specification and the claims which follow, unless the context requires otherwise, the word “comprise,” and variations such as “comprises” and “comprising,” will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of” or “consisting essentially of” to vary scopes of the disclosure.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., nucleic acid sequences encoding DMP8 and mutations thereof), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 1981; 2:482, by the homology alignment algorithm of Needleman & Wunsch, J. Mol. Biol. 1970; 48:443, by the search for similarity method of Pearson & Lipman, Proc. Nat'l. Acad. Sci. USA 1988; 85:2444, by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., 1995 Supplement (Ausubel)).

Examples of algorithms that are suitable for determining percent sequence identity and sequence similarity are the BLAST and BLAST 2.0 algorithms, which are described in Altschul et al., J. Mol. Biol. 1990; 215:403-410 and Altschul et al., Nucleic Acids Res. 1997; 25:3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 1989; 89:10915).

In addition to calculating percent sequence identity, the BLAST algorithm also performs a statistical analysis of the similarity between two sequences (see, e.g., Karlin & Altschul, Proc. Nat'l. Acad. Sci. USA 1993; 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

As used herein, the term “gene” refers to a hereditary unit including a sequence of DNA that occupies a specific location on a chromosome and that contains the genetic instructions for a particular characteristic or trait in an organism.

As used herein, a plant referred to as “haploid” has a single set (genome) of chromosomes and the reduced number of chromosomes (n) in the haploid plant is equal to that of the gamete.

The terms “agronomics”, “agronomic traits”, and “agronomic performance” refer to the traits and underlying genetic elements of a given plant variety that contribute to yield throughout the growing season. Individual agronomic traits include emergence vigor, vegetative vigor, stress tolerance, disease resistance or tolerance, herbicide resistance or tolerance, branching, flowering, seed set, seed size, seed density, standability, threshability, and the like.

As used herein, “breeding” means the genetic manipulation of living organisms.

An “elite line” is an agronomically superior line that has resulted from many cycles of breeding and selection for superior agronomic performance.

The term “germplasm” refers to the genetic material comprising the physical foundation of the hereditary qualities of an organism. As used here, germplasm includes seeds and living tissue from which new plants may be grown, or another plant part, such as a leaf, stem, pollen, or cells, that may be cultured into a whole plant. Germplasm resources provide sources of genetic traits used by plant breeders to improve commercial cultivars.

The term “plant” includes reference to an immature or mature whole plant, including a plant from which seed or grain, or anthers have been removed. The seed or embryo that will produce the plant is also considered to be the plant. Plant parts include leaves, stems, buds, roots, root tips, anthers, seed, grain, embryo, pollen, ovules, flowers, cotyledons, hypocotyls, pods, flowers, shoots and stalks, tissues, cells, and the like.

An “inducer line” is the male parental line used as a source of genes.

The terms “cultivar” and “variety” are used synonymously and mean a group of plants within a species (e.g., Cannabis sativa or subspecies indica or ruderalis) that share certain genetic traits that separate them from the typical form and from other possible varieties within that species.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

The term “genotype” refers to the genetic constitution of a cell or organism.

The term “chromosome segment” designates a contiguous linear span of genomic DNA that resides in planta on a single chromosome.

As used herein, the term “locus” is a defined segment of DNA.

An “allele” is any of one or more alternative forms of a genetic sequence. In a diploid cell or organism, the two alleles of a given sequence typically occupy corresponding loci on a pair of homologous chromosomes.

The term “polymorphism” refers to a change or difference between two related nucleic acids. A “nucleotide polymorphism” refers to a nucleotide that is different in one sequence when compared to a related sequence when the two nucleic acids are aligned for maximal correspondence. A “genetic nucleotide polymorphism” refers to a nucleotide that is different in one sequence when compared to a related sequence when the two nucleic acids are aligned for maximal correspondence, where the two nucleic acids are genetically related, i.e., homologous, for example, where the nucleic acids are isolated from different varieties of a plant, or from different alleles of a single variety, or the like.

A “molecular marker” is a nucleic acid or amino acid sequence that is sufficiently unique to characterize a specific locus on the genome independent of the actual function of its locus. Examples are Restriction Fragment Length Polymorphisms (RFLPs), Single Sequence Repeats (SSRs), Target Region Amplification Polymorphisms (TRAPs), Isozyme Electrophoresis, Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), and Single Nucleotide Polymorphisms (SNPs). All markers are used to define a specific locus on the genome. Each marker is therefore an indicator of a specific segment of DNA, having a unique nucleotide sequence. The positions provide a measure of the relative positions of particular markers concerning one another. When a trait (e.g. the ability to induce haploidy) is stated to be linked to a given marker, it will be understood that the actual DNA segment whose sequence affects the trait generally co-segregates with the marker. More precise and definite localization of a trait can be obtained if markers are identified on both sides (3′ and 5′) of the linear DNA associated with the trait. By measuring the appearance of the marker(s) in progeny of crosses, the existence of the trait can be detected by relatively simple molecular tests without actually evaluating the appearance of the trait itself, which can be difficult and time-consuming because the actual evaluation of the trait requires growing plants to a stage where the trait can be expressed.

The term “amplify” or “amplification” in the context of nucleic acid amplification is any process whereby additional copies of a selected nucleic acid (or a transcribed form thereof) are produced. Typical amplification methods include various polymerase-based replication methods, including the polymerase chain reaction (PCR), ligase-mediated methods such as the ligase chain reaction (LCR), and RNA polymerase-based amplification (e.g., by transcription) methods. An “amplicon” is an amplified nucleic acid, e.g., a nucleic acid that is produced by amplifying a template nucleic acid by any available amplification method (e.g., PCR, LCR, transcription, or the like).

As used herein “one-shot PCR” refers to a multiplex PCR reaction that uses multiple primer sets to amplify several DNA targets simultaneously in a single tube

As used herein “marker assisted selection” refers to the process of selecting a desired trait or desired traits in a plant or plants by detecting one or more molecular markers from the plant, where the molecular marker is linked to the associated or co-segregated desired trait.

The invention provides a method for the rapid molecular screening of F1 hybrids obtained by crossing haploid inducer lines with selected Cannabis varieties. Naturally occurring or mutagenized male lines carrying non-functional mutations at the marker locus (Zhong et al., 2020) can be used to produce maternal haploids or doubled haploids from any heterozygous Cannabis plant, enabling the development of completely homozygous inbred lines in a single generation. The method is applicable to any Cannabis genotype, regardless of nomenclature, and is compatible with diverse commercial varieties and inducer lines (“Inducer K”, “Inducer C”). This scalable, variety or germplasm-independent platform supports breeding objectives including the production of homozygous recombinant inbred lines and the development of true F1 hybrids, thereby enabling the shift from vegetative clonal propagation to stable hybrid seed production in Cannabis.

In one general aspect the application provides a rapid and accurate method for identifying haploid plants derived from crosses between Cannabis donor lines and natural inducer lines carrying spontaneous mutations in the genes involved in egg development including, but not limited to DMP8. The method comprises: (i) whole-genome sequencing of the parental lines; (ii) identification of polymorphic InDel and SNP markers that discriminate maternal and paternal genomes; (iii) design of diagnostic primers targeting InDel polymorphisms; and (iv) one-shot PCR amplification of genomic DNA from F1 progeny to detect the absence of paternal alleles, indicative of haploidy.

In an embodiment of the disclosure, the plant is Cannabis accessions (Sativa, Indica, and Ruderalis).

In an embodiment of the disclosure, the molecular marker is a Single Nucleotide Polymorphism (SNP) marker.

In an embodiment of the disclosure, the molecular marker is an Insertion/Deletion (InDel) marker.

In an embodiment of the disclosure, the molecular marker is a SNP marker and an InDel marker.

In some embodiments, the InDel is at least 4 nucleotides in length.

In some embodiments, the molecular markers are distributed across different chromosomes.

In some embodiments, no additional SNPs or InDels are identified at least 50 nucleotides upstream and downstream of the molecular marker.

In an embodiment of the disclosure, the molecular marker specific to the maternal line is located on chromosome 10, and it is represented by Marker ID 1 (SEQ ID NO: 1).

In an embodiment of the disclosure, the molecular marker specific to the maternal line is located on chromosome 6, and it is represented by Marker ID 2 (SEQ ID NO: 2).

In an embodiment of the disclosure, the molecular marker specific to the paternal line is located on chromosome 3, and it is represented by Marker ID 3 (SEQ ID NO: 3).

In an embodiment of the disclosure, Marker ID 1 falls in locus evm.TU.chr10.1969, which encodes a protein of 448 amino acids.

In an embodiment of the disclosure, Marker ID 2 falls in locus evm.TU.chr6.1735, which encodes a protein of 134 amino acids.

In an embodiment of the disclosure, Marker ID 3 falls in locus evm.TU.chr3.624, which encodes a protein of 368 amino acids.

In an embodiment of the disclosure, an InDel of 11 nucleotides (CCACAACTGTGA; SEQ ID NO: 4), at position 58,833,653 on chromosome 10 induces a frameshift mutation in locus evm.TU.chr10.1969, identifies the maternal genomic material.

In an embodiment of the disclosure, an InDel of 4 nucleotides (CGAGA; SEQ ID NO: 5), at position 85,669,305 on chromosome 6 induces a frameshift mutation in locus evm.TU.chr6.1735, identifies the maternal genomic material.

In an embodiment of the disclosure, an InDel of 23 nucleotides (TAGAAAAATTGTGTCGGGCCAAAC; SEQ ID NO: 6), at position 22,447,076 on chromosome 3 induces a frameshift mutation in locus evm.TU.chr3.624 gene, identifies the paternal genomic material.

In an embodiment of the disclosure, the InDel genotyping primers used are shown in Table 1 below:

TABLE 1
Diagnostic Primer Pairs

| | | | SEQ |
|---|---|---|---|
| | Primer | Sequence | ID NO |
| Marker | Forward 1 | AAGAGGAAAACCACAACTGTGA | 7 |
| ID 1 | Forward 2 | CCACCGTTGCAAAGAGGAAAA | 8 |
| | Common | ACTCTAGCTCGATATGGCGG | 9 |
| | Reverse | | |
| Marker | Forward 1 | CTAAGCTTGCATCCTGCGAGA | 10 |
| ID 2 | Forward 2 | AGTTGGCTAAGCTTGCATCCTG | 11 |
| | Common | TGAGGGTCGAGAGGAATGTTC | 12 |
| | Reverse | | |
| Marker | Forward 1 | CCAACGCTTCAGGACATCAG | 13 |
| ID 3 | Forward 2 | AATTGTGTCGGGCCAAACAG | 14 |
| | Common | CCGCAATTCCAGCATTCCTT | 15 |
| | Reverse | | |

In an embodiment of the disclosure, the genomic sequence surrounding Marker ID 1 is given below:

(SEQ ID NO: 1)
AGGTACACACCAAGGCCAACCACATCAACTTGTTTATCACTTCAATCAC
TCTTCTCTCGGCCTTGGTCTCCCCTCTAACCACCGTGATTTGTACTAAC
ACAACAGCCAATGACGTGAACAATGCAATTGCATTCAATATGAAGAATA
TCTTAAACGACGTGGTGTTCACCATCACCGCCATTCCAGAGTCGTCGTC
GCCTCTAGGGACGGTGAAGATAGCGTCAAAAGCCACCGTTGCAAAGAGG
AAAACCACAACTGTGACTGAGTGGCGTTGTTGATTCCTGCCCTGTGCAG
TTTGCGAAGCTCTTTGGCGCGTTTTTGTTGGTTTTTCGGGTTTGTTCTA
GCTGGATGTGTACGTCCTTTTTGATCTGTGTCACTGTTTTTCTTAGCTC
GTCTCGCGGTTGGTTGAGCTATTTGGCTTTTACGCCGCCATATCGAGCT
AGAGTTTCCTTTATTAGTGATGTTTCTTCGCATAGTGTAAGGCCTTCGG
CTATGTCGAA

In an embodiment of the disclosure, the genomic sequence surrounding Marker ID 2 is given below:

(SEQ ID NO: 2)
TCGGATCTGTTAGACAACCTTAACTTGGAGCATTAGCTACCTTCATTTT
CCTCAGAGGTTTGTTGGCAGTGTTCGTGTTCTTACGTGTAGTAGTTGGA
GTGGTCTTTCTTGGAGTATCTCCTGTGAAACCTTTTCATTTGTCCCTAT
TGCTATTATTATCATAAGGAATAGATTTATCACGAATTTCTTCATCTTG
AGCCATATTCTCTAGCAGAAGTCTAGCCTCCAGTTGGCTAAGCTTGCAT
CCTGCGAGAAAATTCTTCTTGTGCTGCCAATTTTAGCTTGATTTTGGCG
AGTTCTTTGGCCATATCAACGACTGTAGGATTTGGCTTGCAATAATAAT
CATCTTTTTCTTTTTCTTTCCCACCATCTTCAGGGTCTTCGTCTTCGGA
ACATTCCTCTCGACCCTCATTGATGGGCTTTTCATCAACATAATTTTCA
TTACGTTGAGTCTTTTTGGCAGGATGAATAGCTGCTTCAGTTGGATCTC
TGTTGACTCT

In an embodiment of the disclosure, the genomic sequence surrounding Marker ID 3 is given below:

(SEQ ID NO: 3)
TTTGATGAAGCACAAATGGCAAACCTCTTTGCTAGTAATTGAGTTAACC
CAACTACAAAGTTAGTGACAATCTCGGAGGTGAATGGAGCCAAGACAGA
CCCAACGTAGTTAGAGAACCAGTCGTTCATGGGGTCCAAATTTAAGTTC
AAATTTCCTTGACATACTCCTCCTTGCGAGCTACATCCTGATGCTCATT
AATCCAACGCTTCAGGACATCAGATTCCGCAGACCCCTTAGTCTGGGCG
TCCATAGAAAAATTGTGTCGGGCCAAACAGAGTGCAATATGTTTAGGGT
ATGAGTGAACATGAGTCGAAGGAATTGAGGGAACAGGTTGGAATTGCTC
AGCTTTAGAAGGAATGCTGGAATTGCGGGTGCCCTGAGAGGGAGCCTTG
GTGGATTTAGCCTTCTTCAAAGAAGACTCTACAGGAGCCCTCTCCTTCC
TTTTTCCTTTCCTATAGACTAGGGGTTACATCCCGCTCCTTGAGCACAA
CCACCTCCAA

In certain embodiments, the methods further comprise the step of confirming the putative haploid Cannabis plant utilizing genetic techniques. In certain embodiments, the lack of heterozygosity of haploids, which carry only one allele at each locus on different chromosomes, can be used as highly probable evidence of haploidy. In certain embodiments, the SNP genetic analysis is confirmed via DNA sequencing.

In some embodiments, the method further comprises confirming the putative haploid Cannabis plant utilizing flow cytometry.

In an embodiment of the disclosure, the markers specific to the female parent are located on chromosomes 10 and 6, and they are represented by SEQ ID NO: 1 and SEQ ID NO: 2, respectively.

In an embodiment of the disclosure, the marker specific to the male parent is located on chromosome 3, and it is represented by SEQ ID NO: 3.

The following examples of the invention are to further illustrate the nature of the invention. It should be understood that the following examples do not limit the invention and the scope of the invention is to be determined by the appended claims.

### EMBODIMENTS

Embodiment 1 is a method for screening a Cannabis plant obtained by crossing an inducer genotype with a commercial variety for haploidy, wherein the method comprises:
a) sequencing maternal and paternal lines using whole genome sequencing (WGS);
b) identifying contrasting molecular markers between maternal and paternal lines;
c) developing diagnostic primers targeting the molecular marker;
d) screening F1 progeny with one-shot PCR to detect genotype indicative of haploidy.

Embodiment 2 is the method of embodiment 1, wherein the Cannabis plant is selected from the group consisting of Cannabis sativa, Cannabis indica, and Cannabis ruderalis.

Embodiment 3 is the method of embodiment 1, wherein the molecular markers are selected from the group consisting of a Single Nucleotide Polymorphism (SNP) marker, an Insertion/Deletion (InDel) marker, and a combination of both.

Embodiment 4 is the method of embodiment 3, wherein the molecular markers are Insertion/Deletion (InDel) markers.

Embodiment 5 is the method of embodiment 4, wherein the InDel is at least 6 nucleotides in length.

Embodiment 6 is the method of embodiment 3, wherein the molecular markers are distributed across different chromosomes.

Embodiment 7 is the method of embodiment 3, wherein no additional SNPs or InDels are identified at least 50 nucleotides upstream and downstream of the molecular marker.

Embodiment 8 is the method of embodiment 3, wherein the molecular marker specific to the maternal line is located on chromosome 10, and it is represented by Marker ID 1 (SEQ ID NO: 1).

Embodiment 9 is the method of embodiment 3, wherein the molecular marker specific to the maternal line is located on chromosome 6, and it is represented by Marker ID 2 (SEQ ID NO: 2).

Embodiment 10 is the method of embodiment 3, wherein the molecular marker specific to the paternal line is located on chromosome 3, and it is represented by Marker ID 3 (SEQ ID NO: 3).

Embodiment 11 is the method of embodiment 3, wherein the diagnostic primers are selected from the primer pairs as defined in Table 1.

Embodiment 12 is the method of embodiment 1, wherein the method further comprises confirming the putative haploid Cannabis plant utilizing DNA sequencing or flow cytometry.

Various embodiments or subject matter have been described. It will be understood that various modifications may be made without departing from the spirit and scope of the invention. Accordingly, the following examples are intended to illustrate but not limit the scope of inventions described in the claims.

### EXAMPLES

Whole genome sequencing (WGS) of the haploid inducer line (KA-5) and the commercial Cannabis variety (S33) was performed to identify high-confidence insertion/deletion polymorphisms (InDels) distinguishing the two genomes. Quality control was implemented in the pipeline using the tools—FASTQC (Andrews et al. 2021; https://www.bioinformatics.babraham.ac.uk/projects/fastqc/). FASTQ (checked the raw files and provided sequence GC content, overrepresented sequences, and adapter content. After FASTQ (analysis, the reads were trimmed using fastp (Chen et al., Bioinformatics. 2018) and aligned to the reference genome Cannabis sativa cs10 (GCF_900626175) using the BWA-MEM2 (Li et al., arXiv. 2013). The unsorted Sequence Alignment Map (SAM) output files were coordinate-sorted using SAMtools (Danecek et al. Gigascience. 2021) and compressed into Binary Alignment Map (BAM) files. Then, duplicated reads were tagged using MarkDuplicates implemented in Picard https://broadinstitute.github.io/picard/). Clean BAM files were finally used to annotate SNP and InDels in the sample of interest using freebayes variant caller (Garrison et al. arXiv. 2012). Raw variants were filtered by quality (q>30 and DP>10) and only homozygous and contrasting SNP and InDels between samples were further analyzed.

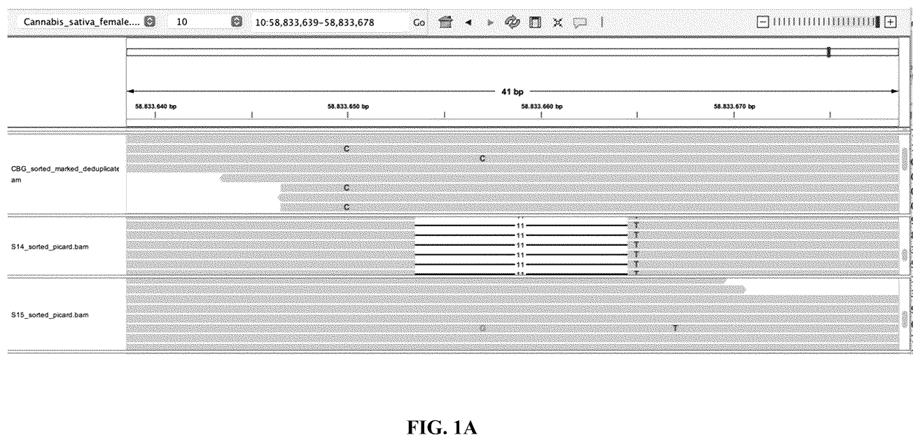

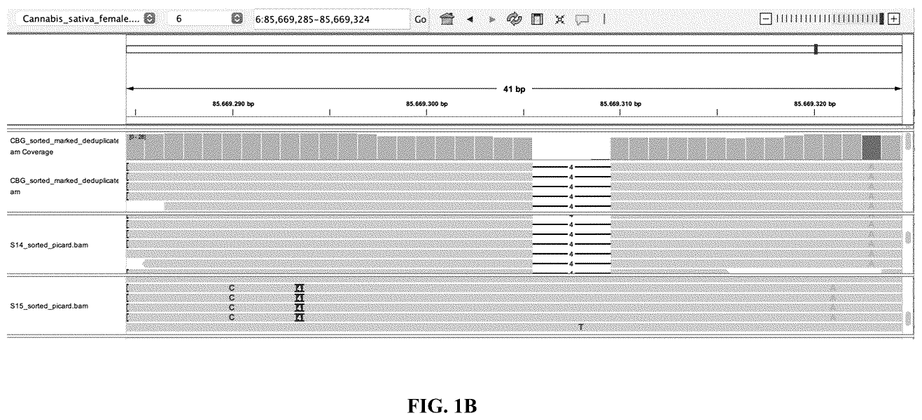

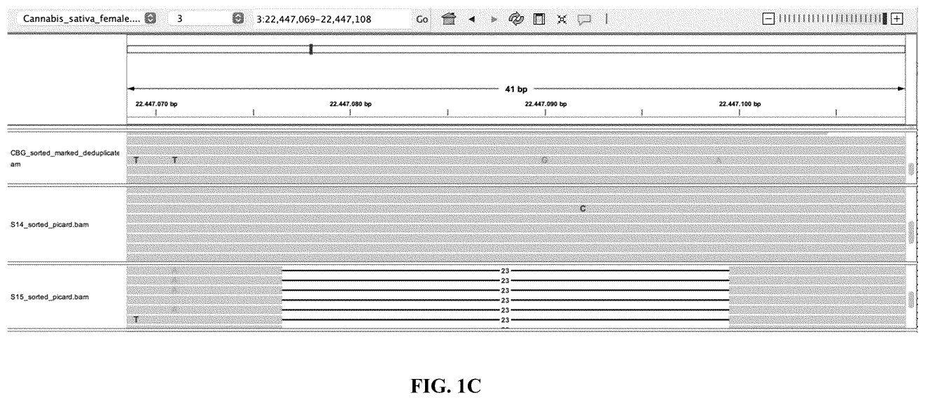

Candidate loci were filtered according to the following criteria: (i) contrasting allelic state between parental genotypes (presence in one parent and absence in the other), (ii) InDel length ≥6 bp to allow unambiguous PCR size discrimination, (iii) absence of additional SNPs or InDels within 50 bp flanking sequences to ensure stable primer annealing, and (iv) distribution across different chromosomes to maximize detection robustness. Three InDel markers were selected, fulfilling the above criteria (FIGS. 1A-1C). Marker ID 1 and Marker ID 2 correspond to deletions present in the paternal haploid inducer but absent in the maternal commercial variety; Marker ID 3 corresponds to a deletion present in the maternal line but absent in the haploid inducer.

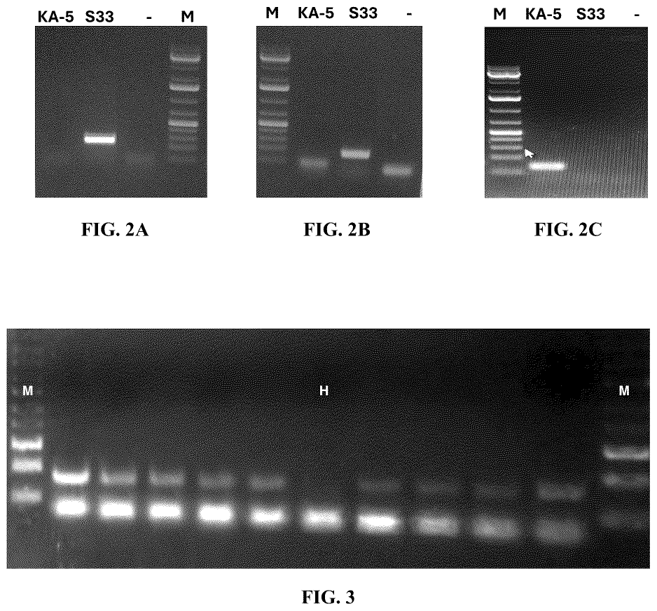

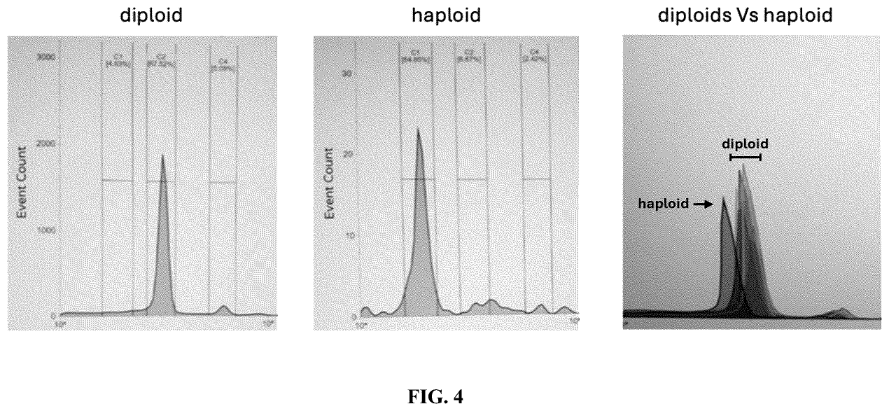

Specific PCR primers were then designed flanking each InDel to generate diagnostic amplicons with differences easily visible on standard agarose gels. The primer sets were optimized to allow multiplex amplification in a single PCR reaction (“one-shot PCR”), thereby simultaneously interrogating multiple genomic regions for parental allele presence. In the one-shot PCR assay (FIGS. 2A-2C), F1 diploids yielded amplification products for both maternal- and paternal-specific markers, reflecting the presence of both parental genomes. In contrast, putative haploids yielded amplification only for the maternal-specific marker(s) and no product for paternal-specific markers, indicating the absence of paternal alleles (FIG. 3).

Flow cytometry confirmed that individuals lacking paternal amplicons had a DNA content corresponding to 1C, consistent with haploidy, while diploid controls displayed 2C DNA content (FIG. 4). Sequence analysis of multiple heterozygous maternal loci further confirmed complete homozygosity in putative haploids, whereas diploid controls retained heterozygous calls at one or more loci.

1. A method for screening a Cannabis plant obtained by crossing an inducer genotype with a commercial variety for haploidy, wherein the method comprises:
a) sequencing maternal and paternal lines using whole genome sequencing (WGS);
b) identifying contrasting molecular markers between maternal and paternal lines;
c) developing diagnostic primers targeting the molecular marker;
d) screening F1 progeny with one-shot PCR to detect genotype indicative of haploidy.

2. The method of claim 1, wherein the Cannabis plant is selected from the group consisting of Cannabis sativa, Cannabis indica, and Cannabis ruderalis.

3. The method of claim 1, wherein the molecular markers are selected from the group consisting of a Single Nucleotide Polymorphism (SNP) marker, an Insertion/Deletion (InDel) marker, and a combination of both.

4. The method of claim 3, wherein the molecular markers are Insertion/Deletion (InDel) markers.

5. The method of claim 4, wherein the InDel is at least 6 nucleotides in length.

6. The method of claim 3, wherein the molecular markers are distributed across different chromosomes.

7. The method of claim 3, wherein no additional SNPs or InDels are identified at least 50 nucleotides upstream and downstream of the molecular marker.

8. The method of claim 3, wherein the molecular marker specific to the maternal line is located on chromosome 10, and it is represented by Marker ID 1 (SEQ ID NO: 1).

9. The method of claim 3, wherein the molecular marker specific to the maternal line is located on chromosome 6, and it is represented by Marker ID 2 (SEQ ID NO: 2).

10. The method of claim 3, wherein the molecular marker specific to the paternal line is located on chromosome 3, and it is represented by Marker ID 3 (SEQ ID NO: 3).

11. The method of claim 3, wherein the diagnostic primers are selected from the primer pairs as defined in Table 1.

12. The method of claim 1, wherein the method further comprises confirming the putative haploid Cannabis plant utilizing DNA sequencing or flow cytometry.
